(19) United States
(12) Patent Application Publication
(10) Pub. No.: US 2012/0253384 A1
(43) Pub. Date: Oct. 4, 2012

(54) Nicy migraine hugger

(76) Inventors: Cynthia Lee Bosnyak, Hemlock, MI (US); Tina Marie Bosnyak, Covina, CA (US)

(21) Appl. No.: 13/065,775

(22) Filed: Mar. 29, 2011

(51) Int. Cl. A61B 19/00; A61F 7/10

(52) U.S. Cl. 606/204.15; 607/109

(57) ABSTRACT

“Nicy Migraine Hugger”, will help assist the many millions of people, who suffer from pains, associated with headaches and migraines, until they are able to reach a more permanent solution. The hugger's pressure and coolness, works together to reach the main nerve points of the head, where the pain from headaches and migraines are. It fits nicely around the whole hairline, and will fit under or on top of the hair.

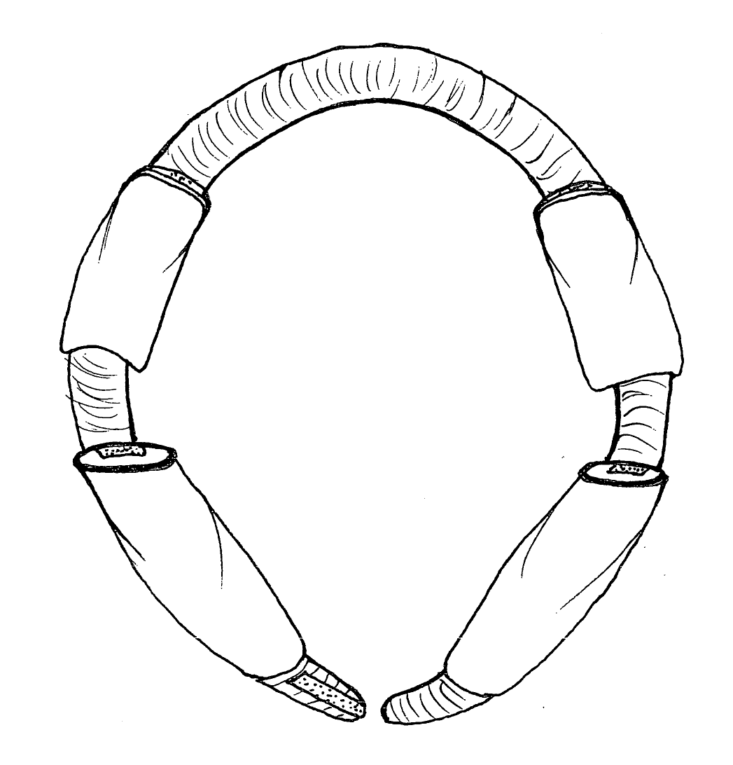

The hugger is comfortable, because it is supported by four pouches, located under the flexible liner. The liner can be bended, which makes it possible for it to be turned into many directions. Since this product is flexible, it will package easily, in addition, it weighs only a few ounces, therefore making is less expensive to ship. The total expenses to make this hugger is a comparable advantage, because the average consumer will benefit as they will be able to afford to own one.

### BACKGROUND

The idea for “NICY MIGRAINE HUGGER” came from the experience of migraines I suffered with for over 40 years now. Being sensitive to drugs, I knew I had to come up with something to reduce the pain I was enduring. Most of the ice and gel wraps and bands on the market today do not stay cold for long and they look more like a [knight in shinning armor wrap]. I wanted to create something that could be worn either on top or under the hair, without looking like I just got out of the hospital, (as shown in the drawing). It's not like I want the whole world to see how obvious it was, that I was suffering with head pains. It's exhausting to suffer with head pain and look goofy at the same time.

I knew I could create a flexible, a bendable, a comfortable and a safe hugger, that would stay colder longer than some of the headache reducer, relief wraps or bands that are on the market. To be able to hide the hugger under the hair is a [plus]. It took a few months, but I continued to think of light weight and thin types of materials that would help keep the hugger a great product, yet have the ability to be small enough to hide under my hair. Making NICY MIGRAINE HUGGER, was built on many attributes of pressure, coolness time, comfort, flexibility and reasonable pricing. After testing NICY, we noticed the hugger can assist more than just people with migraines or headaches. My sister (who is the additional inventor), tested our invention. Although she does not suffer with migraines, she claims it relieves some stress when she wears it after a long day of work. It fits completely around the hairline and hooks in the back, if preferred that way. Since it's flexible, it can bend and fasten in many ways.

Not to dismiss any attributes that many products on the market has, but they just do not satisfy as many attributes as our NICY MIGRAINE HUGGER does. Months of research we did, made us aware other problems people are experiencing with some other ice or gel wraps, bands, or caps. In order for the hugger to become more useful, we kept improving our NICY MIGRAINE HUGGER. The hugger now holds coolness for longer periods of time; it has the ability to hide it under hair; it is flexible enough to bend, in order to tighten or loosen up it's grip. Since some people need to interchange pouches with hot and cold, I made the two bottom pouches (as shown towards the bottom of the drawing), big enough to fit small heatmax packs. [The drawing is drawn from the inside view of our hugger].

Two smaller pouches are aligned with the eyebrows and against the temples (as shown towards the top of the drawing). A lot of pain is within the eyebrow areas, so the hugger fits snuggly all around the whole hairline. Enough pressure is possible by the user being able to adjust the hugger by tightening or loosening it. The amount of pressure applied, is up to the user. The Velcro connects at the bottom of skull, which can tighten or loosen the hugger, (as shown towards the bottom of the drawing, where the dots are). The tighter the hugger is fastened, the colder and more pressure is sent around important nerve areas. The pouches are located on the inside of the hugger, which helps the coldness to touch the head more directly, and acts like a cushion between the liner and the head.

### MATERIALS & FUNCTIONS

NICY MIGRAINE HUGGER, is oval-circular after bent around the hairline, [as shown in the drawing]. Velcro pieces are placed at bottom on each end, which allows the hugger to fit snuggly around the hairline. It is an adjustable, bendable liner because it is flexible. The wire inside the vinyl tube, covered with Velcro, is [non-obvious], is safe, and is safely guarded against becoming uncomfortable. The ends of the vinyl tube were melted and clamped shut. Inside the ends of the vinyl tube, [inwards to about an inch], is a small piece of 2× stronger Velcro, which is used to keep the wire from moving out the ends, during bending. Then Velcro was placed around the vinyl tube to cushion the liner, with the sticky side of the velcro facing outwards. Foil tape was then wrapped around the Velcro to keep it from unraveling, but allows enough adjusted movement in order to bend the hugger into other shapes.

There are four pouches cut and sewn within the cover, which is made up of soft stretch material. A very small hem is sewn around the edges of that soft, stretch, cover, material. The four pouches include two pouches smaller and two pouches bigger. These four pouches are facing the inwards part of the liner. The pouches blend long-ways, along side of the liner (as shown in the drawing). The tops of the pouches, which are later filled with the baggies (of frozen vegetables, or small heat pads), can be sealed with small pieces of velcro, one piece on each side of the top opening. The cover is made of one piece, and the pouches are measured to fit accordingly, while keeping the liner's width as small as possible. All the pouches are (aligned with cloth duct tape) to improve the coldness stages, and the cloth duct tape also protects the insides of the pouches in case the baggies sweat a little, (if need be), from the frozen vegetables added.

The two smaller pouch's position aligns with the each eyebrow as it sits snuggly against the temples (as shown towards the top of the drawing). The two bigger pouches are at the bottom of the skull (as shown towards the bottom of the drawing). The bottom pouches can be fastened close enough to each other so that it [improves coldness to stay for a longer period of time]. The bigger bottom pouches holds coolness longer than the smaller pouches, but the head remains cool even when the temple pouches have less coolness, as it lingers onto more cooling time. The assistance of the two smaller pouches by the temples (aligned with duct tape), together with the hugger's pressure, materials used, and the filled baggies with frozen peas [or frozen mixed vegetables], eases into starting the coldness cycle. After the hugger becomes warmer, the coolness lingers throughout the head for a while. Since the hugger is comfortable, it can be worn for a long period of time, and can be turned around to position the [bigger pouches over the eyes].

The hugger can be moved over the top of the head as it is flexible. If it is worn above the ears, instead of behind the ears (around the hairline), it can serve as a tool to bring relief around the eyes or just to cool the top area of the head. Cold temperatures help to reduce swelling. Heat pads also can be inserted into the pouches by choice, if the user prefers heat. When the hugger is first put on, you can feel the coldness coming through all the layers of materials used. The pressure of the hugger (as the user can adjust), keeps those nerves covered comfortably. Filling the baggies with frozen peas or frozen mixed vegetables is [non-toxic].

The user can take the options to fill the hugger with their choices of frozen vegetables by [simply] using a small kitchen spoon to pour peas into the baggies, then [simply] turning the spoon around to slowly stuff the filled baggies into each desired pouch, which are aligned with cloth duct tape. After the hugger's baggies are filled and put in the pouches, the user can [simply throw the whole hugger into the freezer]. The hugger, will then be ready after freezing, to be put on and used [immediately] upon the first sign of head pain or strain. It is up to the user to decide if they prefer a cold or a warmer hugger, depending how each feels to them. The hugger stays colder longer if you allow it to [completely freeze] for two hours. If you take it out before it is frozen, it will be cold, but not frozen, which reduces the time it remains cool. If the hugger is frozen, there is a better chance that it can help reduce and give relief to the pain, [until it is possible to reach a more permanent solution].

If the head pain or migraine is not address right away, it can lead to an increased chance of accumulating into a full-blown, migraine or headache, which is extremely painful and can last from hours to days. This hugger can stay colder longer than a lot of the average head pain reducer wrap, cap, or bands on today's market, if it is used and worn correctly. It is comfortable enough to wear for an extended amount of time. Although the liner is made from wire and vinyl tubing, the velcro covers the liner and the soft stretch material added, reduces hard impacts against the head. The Velcro's sticky side is on the outside then foil tape is applied around the Velcro, to keep the Velcro, from unraveling. The foil helps keep the liner cooler also. [The inside pouches also serves as a cushion against the head], because within minutes the filled baggies begin to soften. The color, design and type of stretched material of the cover, will be up to the user to choose from and to decide which option they prefer. The little baggies will be included therefore, all the user has to do is to fill them and insert them into the pouches, then close the top of the pouch shut, with the velcro that attaches at the top of the pouches.

### MULTI-USES:

The many months of testing processes proved to find that NICY MIGRAINE HUGGER might be able to serve as a cooling item also. The hugger can keep a head cool and can be worn in the summertime, if someone is overcome with too much heat. It can [conveniently] be thrown into a cooler chest, as people are going on their picnics or any other type of outdoor activity. The hugger is safe for children.

NICY MIGRAINE HUGGER, can relieve some stress, by the pressure it gives around the hairline of the head. After a long day at work, or just after a long day, the pressure of the hugger can relax those head muscles. [The hugger fits along side of the hairline], although it can fit elsewhere, because it is flexible. Therefore, can bend in other directions, it can be used to wrap around other areas of the body, like turning it around, placing the bigger pouches over the eyes. Although some people might prefer heat, coldness helps to reduce the speeds of pulsating pains from nerves.
(Google 2011)

### CONCLUSION OF SPECIFICATIONS AND ATTRIBUTES

Our NICY MIGRAINE HUGGER, weighs only a few ounces, yet contains pressure options available for each individual's needs and/or wants. [Packaging our invention will be quite easy] because the hugger is safe to flex, and can be bent in different directions. As mentioned previously, it only weighs a few ounces, which can reduce the cost to ship and reduce the total cost of this product. The comparable cost of the hugger, being more inexpensive to make, will therefore be comparably affordable enough, for the average person to be able to buy. Once someone tries on the hugger, [they will not want to take it off] because even after the coolness subsides, the comfortable pressure feeling around the head's hairline, remains constant.

Although one size does not fit all, the Velcro allows the hugger to be adjusted a few inches. That means that a small, medium, large or extra large size will be sufficient enough to accommodate most head sizes. Furthermore, since the width of the hugger is small, it is possible to wear the hugger under a few inches of hair, if an individual prefers to hide it. And glasses can be worn with the hugger, because the pouches are above and below the area where glasses hook.

I claim:

1). NICY MIGRAINE HUGGER'S pressure, helps to reduce and relieve some of the pains and stresses that are associated with headaches and/or migraines.

2). NICY MIGRAINE HUGGER'S materials helps it to stay cooler longer than a lot of the average gelled, head masks or migraine wraps, that are presently on the market.

3). NICY MIGRAINE HUGGER is flexible.
